(19) United States
(12) Patent Application Publication
(10) Pub. No.: US 2017/0266465 A1
(43) Pub. Date: Sep. 21, 2017

(54) METHOD AND SYSTEM OF MANIPULATING BILAYER MEMBRANES

(71) Applicant: Technion Research & Development Foundation Limited, Haifa (IL)

(72) Inventors: Eitan KIMMEL, Ramat-HaSharon (IL); Shy SHOHAM, Rehovot (IL); Boris KRASOVITSKI, Nesher (IL)

(73) Assignee: Technion Research & Development Foundation Limited, Haifa (IL)

(21) Appl. No.: 15/607,727

(22) Filed: May 30, 2017

Related U.S. Application Data
(63) Continuation of application No. 13/696,098, filed on Nov. 5, 2012.
(60) Provisional application No. 61/331,451, filed on May 5, 2010.
(60) Provisional application No. 61/364,471, filed on Jul. 15, 2010.

(51) Int. Cl. A61N 7/00; A61M 37/00

(52) U.S. Cl. CPC A61N 7/00; A61M 37/0092; A61N 2007/0039; A61B 8/0808

(57) ABSTRACT

A method of changing the volume of an intra-bilayer membrane space of at least one bilayer membranous structure of a target tissue. The method comprises providing at least one characteristic of a target tissue having at least one bilayer membranous structure, selecting an acoustic energy transmission pattern set to change a volume of an intra-bilayer membrane space of a bilayer membrane of the at least one bilayer membranous structure according to the at least one characteristic, and applying acoustic energy on the target tissue according to the selected acoustic energy transmission pattern.

### RELATED APPLICATIONS

This application is a Continuation of U.S. patent application Ser. No. 13/696,098 filed on Nov. 5, 2012, which is a National Phase of PCT Patent Application No. PCT/IL2011/000359 having International Filing Date of May 5, 2011, which claims the benefit of U.S. Provisional Patent Application Nos. 61/331,451 filed on May 5, 2010 and 61/364,471 filed on Jul. 15, 2010. The contents of the above applications are all incorporated by reference as if fully set forth herein in their entirety.

### FIELD AND BACKGROUND OF THE INVENTION

The present invention, in some embodiments thereof, relates to method and system of manipulating bilayer membranes and, more particularly, but not exclusively, to method and system of manipulating bilayer membranes using acoustic energy.

Ultrasound (US) acoustic energy is used in medicine and biology, where the pressure amplitude (p or p_{A}) ranges from O(10⁴) Pascal (Pa) low intensity US to O(10⁵) Pa used in short bursts for imaging, and up to O(10⁶) Pa and even O(10⁷) Pa in high intensity focused ultrasound (HIFU) applications. The amplitude of the above pressure range is between about O(10⁴) and O(10⁷) Pa with power intensity (I) between O(10)⁻² and O(10⁴) Wcm⁻², where for a propagating wave I=p²/2ρc where ρ denotes medium density and c denotes speed of sound. Note that the frequency (f) range lies between 0.02 Megahertz (MHz) and 30 MHz. When acoustic energy is applied for therapeutic purposes, cavitation is performed whereas the acoustic gas bubble interacts with cells, tissue and organ, see Carstensen, E. L., S. Gracewski, et al. (2000). “The search for cavitation in vivo.” Ultrasound in Medicine and Biology 26(9): 1377-1385, which is incorporated herein by reference. As used herein cavitation means an activity of gas bubbles in the US field where the bubbles are formed from gas pockets known as cavitation nuclei, steady pulsations (stable cavitation) and possible collapse (transient cavitation), see Leighton, T. G. (1997). The Acoustic Bubble. San Diego—London, Academic Press, which is incorporated herein by reference.

When acoustic energy is applied for imaging, safety is achieved by avoiding cavitation. Common US bioeffects in high US intensity include for instance lysis of red blood cells (RBC) in vitro, see Carstensen, E. L., P. Kelly, et al. (1993). “Lysis of Erythrocytes by Exposure to CW Ultrasound.” Ultrasound in Medicine and Biology 19(2): 147-165, which is incorporated herein by reference, damage to blood vessels and hemorrhage, see Child, S. Z., C. L. Hartman, et al. (1990). “Lung Damage from Exposure to Pulsed Ultrasound.” Ultrasound in Medicine and Biology 16(8): 817-825, which is incorporated herein by reference and US enhanced permeability, which may by incorporated herein by reference, Tezel, A. and S. Mitragotri (2003). “Interactions of inertial cavitation bubbles with stratum corneum lipid bilayers during low-frequency sonophoresis” Biophysical Journal 85(6): 3502-3512, which is incorporated herein by reference. These US induced bioeffects are attributed to bubble activity held externally to cells and exert pressure thereon by forming bubbles in proximity to solid cellular surfaces such as the epithelium or endothelium, see Tezel, A. and S. Mitragotri (2003). “Interactions of inertial cavitation bubbles with stratum corneum lipid bilayers during low-frequency sonophoresis.” Biophysical Journal 85(6): 3502-3512, Krasovitski, B. and E. Kimmel (2004). “Shear stress induced by a gas bubble pulsating in an ultrasonic field near a wall.” IEEE Transactions on Ultrasonics Ferroelectrics and Frequency Control 51(8): 973-97, and Marmottant, P. and S. Hilgenfeldt (2003). “Controlled vesicle deformation and lysis by single oscillating bubbles.” Nature 423(6936): 153-156, which are incorporated herein by reference.

Evidences show that such bioeffects intensify whenever encapsulated microbubbles with diameters of a few micrometers, known also as ultrasound contrast agents (UCAs) are used as enhancers of ultrasound scattering for imaging of blood vessels after being introduced intravenously into the blood circulation. The presence of UCAs in the blood circulation increases the level of damage to blood vessels and hemorrhage in vivo Skyba, D. M., R. J. Price, et al. (1998). “Direct in vivo visualization of intravascular destruction of microbubbles by ultrasound and its local effects on tissue.” Circulation 98(4): 290-293, which is incorporated herein by reference. Similarly, in vitro, the response of cells is amplified by the presence of UCAs in proximity to the cells Postema, M., A. Van Wamel, et al. (2004). “Ultrasound-induced encapsulated microbubble phenomena.” Ultrasound in Medicine and Biology 30(6): 827-840, which is incorporated herein by reference.

Methods of effecting cell functioning, without cavitations, using low intensity US energy are described in Carstensen, E. L., S. Gracewski, et al. (2000). “The search for cavitation in vivo.” Ultrasound in Medicine and Biology 26(9): 1377-1385 and in Tyler, W. J., Y. Tufail, et al. (2008). “Remote Excitation of Neuronal Circuits Using Low-Intensity, Low-Frequency Ultrasound.” Plos One 3(10), which are incorporated herein by reference. In Tyler, remote excitation of neuronal circuits is induced by low intensity US.

### SUMMARY OF THE INVENTION

According to some embodiments of the present invention there is provided a method of changing the volume of an intra-bilayer membrane space of at least one bilayer membranous structure. The method comprise providing at least one characteristic of the at least one bilayer membranous structure, selecting an acoustic energy transmission pattern set to change a volume of an intra-bilayer membrane space of a bilayer membrane of the at least one bilayer membranous structure according to the at least one characteristic, and applying acoustic energy on the target tissue according to the selected acoustic energy transmission pattern.

Optionally, the at least one bilayer membranous structure is at least one cell, the providing comprising providing at least one characteristic of a target tissue having the target at least one cell.

Optionally, the at least one bilayer membranous structure comprises at least one membranous delivery vessel, the providing comprising providing at least one characteristic of a target tissue having the target at least one bilayer membranous structure.

Optionally, the at least one bilayer membranous structure is a member of a group consisting of a cell, a cell organelles, a membranous delivery vessel, a liposome, and any microorganism encapsulated by a bilayer membrane.

More optionally, the selecting is performed according to at least one desired bioeffect on the target tissue.

More optionally, the method further comprises directing at least one acoustic energy source in front of the target tissue according to the selected acoustic energy transmission pattern and using the at least one acoustic energy source for performing the applying.

Optionally, the acoustic energy transmission pattern defines a plurality of sequential acoustic energy transmission cycles.

More optionally, each acoustic energy transmission cycle, apart from the first of the plurality of sequential acoustic energy transmission cycles have a higher frequency than another the acoustic energy transmission cycle.

Optionally, the selecting comprises selecting at least one member of a group consisting of: a frequency of an acoustic energy transmission, a transmission power of the acoustic energy transmission, a transmission angle of the acoustic energy transmission, and a transmission interlude according to the at least one characteristic.

Optionally, the selecting estimating at least one of attraction force and repulsion force between leaflets of the intra-bilayer membrane.

Optionally, the selecting is performed according to a desired increment in the volume of the intra-bilayer membrane space.

Optionally, the selecting comprises estimating the volume of a pulsating gas bubble generated by acoustic energy transmission energy according to the at least one characteristic and selecting the acoustic energy transmission pattern according to the volume.

More optionally, the applying is performed to induce cell necrosis in the target tissue.

More optionally, the applying is being performed to change a rate of introducing exogenous material into the intra cellular space of cells of the target tissue.

Optionally, the applying is performed to stimulate at least one cellular process in the target tissue.

Optionally, the applying is performed to slow down at least one cellular process in the target tissue.

More optionally, the applying is performed to change at least one mechanical characteristic of at least one bilayer membranous structure of the target tissue.

Optionally, a frequency of the acoustic energy is between 0.1 MHz and 30 MHz.

Optionally, an amplitude of a pressure applied by the acoustic energy on the bilayer membrane is about 0.1 megapascal (MPa)

Optionally, the volume is defined between trans-membrane proteins connecting leaflets of the bilayer membrane.

Optionally, the applying comprises forming at least one hydrophilic passage passing through a plurality of leaflets of the bilayer membrane.

Optionally, the acoustic energy includes ultrasound (US) acoustic energy.

Optionally, the acoustic energy includes acoustic shock wave transmission.

According to some embodiments of the present invention there is provided a system of changing the volume of an intra-bilayer membrane space of at least one bilayer membranous structure. The system comprises an interface which provides at least one characteristic of a target tissue having at least one bilayer membranous structure, a computing unit which selects an acoustic energy transmission pattern set to change the volume of an intra-bilayer membrane space of the at least one bilayer membranous structure according to the at least one characteristic, and a controller which instructs an acoustic energy source to apply acoustic energy on the target tissue according to the selected acoustic energy transmission pattern.

Optionally, the interface comprises a man machine interface for allowing a user to select at least one desired bioeffect, the computing unit selecting the acoustic energy transmission pattern according to the at least one desired bioeffect.

More optionally, the at least one desired bioeffect is a member of a group consisting of: changing a rate of introducing exogenous material into the intra cellular space of cells of the target tissue, stimulating at least one cellular process in the target tissue, inhibiting at least one cellular process in the target tissue, and changing at least one mechanical characteristic of at least one bilayer membranous structure of the target tissue.

Optionally, the system further comprises a database hosting a plurality of acoustic energy transmission patterns, the computing unit selects the acoustic energy transmission pattern from the database.

According to some embodiments of the present invention there is provided a method of operating at least one acoustic energy source for changing the volume of an intra-bilayer membrane space of at least one bilayer membranous structure. The method comprises receiving at least one characteristic of one or more of at least one bilayer membranous structure, a target tissue having the at least one bilayer membranous structure, and at least one tissue surrounding the at least one bilayer membranous structure, selecting an acoustic energy transmission pattern set to change the volume of an intra-bilayer membrane space of the at least one bilayer membranous structure according to the at least one characteristic, and instructing the at least one acoustic energy source to apply acoustic energy on the target tissue according to the selected acoustic energy transmission pattern.

Optionally, the selecting is performed so that the applying of acoustic energy according to the acoustic energy transmission pattern on the at least one bilayer membranous structure induce at least one rupture thereon.

Optionally, the instructing is set to induce a release of at least one medicament from the at least one bilayer membranous structure.

According to some embodiments of the present invention there is provided a method of estimating a safety level of at least one acoustic energy transmission. The method comprises providing at least one characteristic of a target tissue having a plurality of cells, providing at least one transmission characteristic of an acoustic energy transmission for radiating the target tissue, estimating an increment in the volume of an intra-bilayer membrane space of the plurality of cells in response to the acoustic energy transmission, computing a safety level according to the increment, and outputting a notification indicative of the safety level.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

Implementation of the method and/or system of embodiments of the invention can involve performing or completing selected tasks manually, automatically, or a combination thereof. Moreover, according to actual instrumentation and equipment of embodiments of the method and/or system of the invention, several selected tasks could be implemented by hardware, by software or by firmware or by a combination thereof using an operating system.

For example, hardware for performing selected tasks according to embodiments of the invention could be implemented as a chip or a circuit. As software, selected tasks according to embodiments of the invention could be implemented as a plurality of software instructions being executed by a computer using any suitable operating system. In an exemplary embodiment of the invention, one or more tasks according to exemplary embodiments of method and/or system as described herein are performed by a data processor, such as a computing platform for executing a plurality of instructions. Optionally, the data processor includes a volatile memory for storing instructions and/or data and/or a non-volatile storage, for example, a magnetic hard-disk and/or removable media, for storing instructions and/or data. Optionally, a network connection is provided as well. A display and/or a user input device such as a keyboard or mouse are optionally provided as well.

### BRIEF DESCRIPTION OF THE SEVERAL VIEWS OF THE DRAWINGS

The patent or application file contains at least one drawing executed in color. Copies of this patent or patent application publication with color drawings will be provided by the Office upon request and payment of the necessary fee.

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIG. 1 is a flowchart of a method of changing the volume of intra bilayer membrane space of bilayer membranous structures using acoustic energy, according to some embodiments of the present invention;

FIG. 2 is a schematic illustration of a model of a lipid bilayer membrane having two substantially flat, parallel, monolayer leaflets with an intra-bilayer membrane hydrophobic space, according to some embodiments of the present invention;

FIG. 3 is a schematic illustration of a lipid bilayer membrane model having an intra-bilayer membrane space with an expended volume, according to some embodiments of the present invention;

FIG. 4A is an exemplary bilayer membrane, according to some embodiments of the present invention;

FIGS. 4B-4E are schematic illustrations of different bioeffects to the leaflets of the bilayer membrane, according to some embodiments of the present invention;

FIGS. 5A and 5B which are schematic illustrations of a simplified model of a cell and a cell with expended intra-bilayer membrane space;

FIG. 6, which is a schematic illustration of a system that applies acoustic energy for changing the volume of intra bilayer membrane space of bilayer membranous structures of a target biological tissue, according to some embodiments of the present invention;

FIG. 7 is a method of estimating the safety of an acoustic energy transmission, according to some embodiments of the present invention;

FIGS. 8A and 8B are graphs of the dynamic response of the bilayer membrane and the tissue around it as predicted by a simulation for four exemplary initial cycles of exposure to continuous wave acoustic energy;

FIGS. 8C-8E depict an actual pressure pulse and amplification applied on a wall membrane by an exemplary bubble and the effect of the distance between the center of the bubble and the membrane wall, according to some embodiments of the present invention; and

FIGS. 9A-9G are images of the bioeffects of acoustic energy transmissions on a fish skin tissue.

### DESCRIPTION OF SPECIFIC EMBODIMENTS OF THE INVENTION

The present invention, in some embodiments thereof, relates to method and system of manipulating bilayer membranes and, more particularly, but not exclusively, to method and system of manipulating bilayer membranes using acoustic energy.

According to some embodiments of the present invention there is provided a method and a system of changing the volume of an intra-bilayer membrane space using acoustic energy. The intra-bilayer membrane space may be of cellular membranes of one or more bilayer membranous structures of a target biological tissue, artificial membranes of bilayer membranous structures, organelles, for example the nucleus, mitochondria, and/or endoplasmic reticulum, microbes, microorganisms, and/or liposomes. The method and system may be used for generating desired bioeffects in a target biological tissue, for example creating pores or ruptures in the bilayer membranous structures bilayer membranes for changing a rate of introducing exogenous material into the intra bilayer membranous structure space, such as cellular space (cytoplasm), stimulating and/or inhibiting one or more cellular processes, and/or changing one or more mechanical characteristics of the cells. The method and system may be used for releasing content of membranous delivery vessels having a bilayer membrane, for example for releasing medicaments at a desired venue and/or timing in the body. Such a release mechanism may be generated by transmitting an acoustic energy having amplitude, frequency and/or phase which is set to create pores and/or ruptures in the bilayer membrane of the vessels.

Optionally, one or more characteristics of a target biological cellular and/or artificial tissue are provided, for example manually by a user or automatically from a diagnosis system or a database. These characteristics allow selecting an acoustic energy transmission pattern set to change the volume of the intra-bilayer membrane space of the target tissue. Acoustic energy is applied on the target biological and/or artificial tissue, referred to herein as a target tissue, according to the selected acoustic energy transmission pattern, causing one or more desired bioeffects.

According to some embodiments of the present invention there is provided a system of changing the volume of intra-bilayer membrane space of bilayer membranous structures of a target tissue, such as cells, cell organelles, for example the nucleus, mitochondria, and/or endoplasmic reticulum, membranous delivery vessels, structures having artificial membrane based elements such as liposomes, and microorganisms, such as Bactria. The system is based on an interface which allows providing one or more characteristics are outlined, a computing unit which selects an acoustic energy transmission pattern according to the characteristics and a controller which instructs an acoustic energy source, such as an US source, for example an array of US transducers or an acoustic shock waves generator, for example an electrical spark discharge, to apply acoustic energy on the target tissue according to the selected acoustic energy transmission pattern.

Before explaining at least one embodiment of the invention in detail, it is to be understood that the invention is not necessarily limited in its application to the details of construction and the arrangement of the components and/or methods set forth in the following description and/or illustrated in the drawings and/or the Examples. The invention is capable of other embodiments or of being practiced or carried out in various ways.

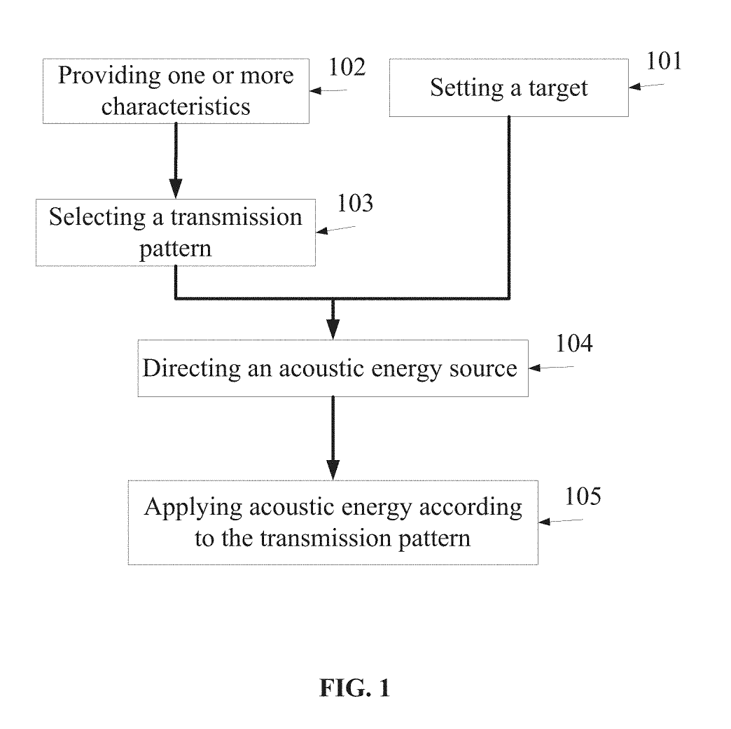

Reference is now made to FIG. 1, which is a flowchart of a method 100 of changing the volume of intra bilayer membrane space of bilayer membranous structures using acoustic energy, such as ultrasound (US) acoustic energy and/or acoustic shock waves, according to some embodiments of the present invention. The bilayer membranous structures may be cells with bilayer membranes of a target biological tissue, which are susceptible to US stimulation. For example, the target biological tissue includes a cluster of cells each having a cellular bilayer membrane that encloses a nucleus and/or other organelles in the cytoplasm and/or a cluster of cells each having an artificial lipid bilayer membrane. The target tissue may include a portion of any epithelia and/or of the stratum corneum of a patient and/or an inner tissue, such as the keratinocyte layer, the stratum lucidum, the stratum granulosum, and/or any inner tissue. The method 100 may be used for causing one or more bioeffects in the biological tissue, for example creating pores or ruptures, for brevity referred to herein as ruptures, in the cells' bilayer membranes for changing a rate of introducing exogenous material into the intra cellular space, stimulating and/or inhibiting one or more cellular processes, and/or changing one or more mechanical characteristics of the cells. It should be noted that though most of the description herein refers to a bilayer membrane of a cell of a biological tissue, any bilayer membrane of a bilayer membranous structure may be similarly processed, for example a bilayer membrane of a membranous delivery vessel, an artificial membrane and/or a bilayer membrane of a liposome, a bilayer membrane of an organelle, organelles, for example the nucleus, mitochondria, and/or endoplasmic reticulum, and/or a bilayer membrane of a microorganism, such as Bactria. As further described below, the applying of acoustic energy on a bilayer membrane 200, such as a lipid bilayer membrane, increases the volume of bubbles therein. This may be done by applying acoustic energy in a wide range of US intensities.

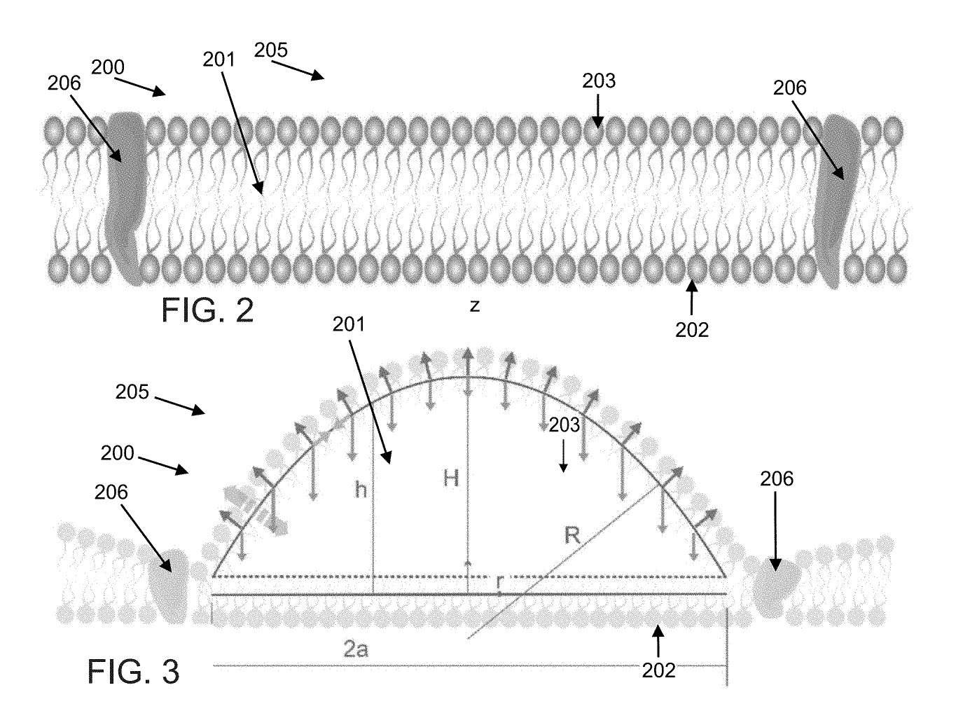

The method allows forming cavitation nuclei in the intra cellular bilayer membrane space of bilayer membrane of cells of a biological tissue. As used herein, cavitation nuclei means inhomogeneity formed in a liquid by bubbles consist at least in part of a volume of gas. For clarity, reference is now made to FIG. 2, which is a schematic illustration of a model of a multi layered epithelium 201, such as a lipid bilayer membrane having two substantially flat, parallel, monolayer leaflets 202, 203 with an intra-bilayer membrane hydrophobic space 201 between them. Optionally, aqueous solution 205, such as water, surrounds the lipid bilayer membrane from the external hydrophilic side 203 and gas molecules that are dissolved in the water pass freely via the leaflets 202, 203 and may be found in the intra-bilayer membrane space. FIG. 2 depicts the lipid bilayer membrane 200 at equilibrium, where no force is acts between the leaflets 202, 203.

Reference is now made to the method of changing the volume of intra bilayer membrane space of cells of a target tissue membranous delivery vessels using acoustic energy. As shown at 101, a target is set, for example by placing a target tissue in a target space, which optionally includes an aqueous solution, such as water, injecting membranous delivery vessels to a patient, and/or placing artificial tissue having bilayer membrane element in a target area. If the target tissue is a body tissue, the patient may be placed in a designated location, for example positioned horizontally on a bed, to allow an acoustic energy source to transmit acoustic energy onto the target tissue. It should be noted that the acoustic energy source may be any acoustic energy source, for example acoustic energy sources that combine other probes, acoustic energy source which generate focused and/or controlled ultrasonic beams and the like.

If the target is releasing the content of membranous delivery vessels having a bilayer membrane, for example for releasing medicaments at a desired venue and/or timing in the body, the acoustic source may be placed to radiate a certain target bodily region and/or organ so that the membranous delivery vessels are radiated only when is at the target bodily region and/or organ. In such a manner, the acoustic energy, which is optionally set with an amplitude, frequency and/or phase set to create pores and/or ruptures in the bilayer membrane of the vessels, induce the release of the medicaments only at the target bodily region and/or only when the acoustic energy is active.

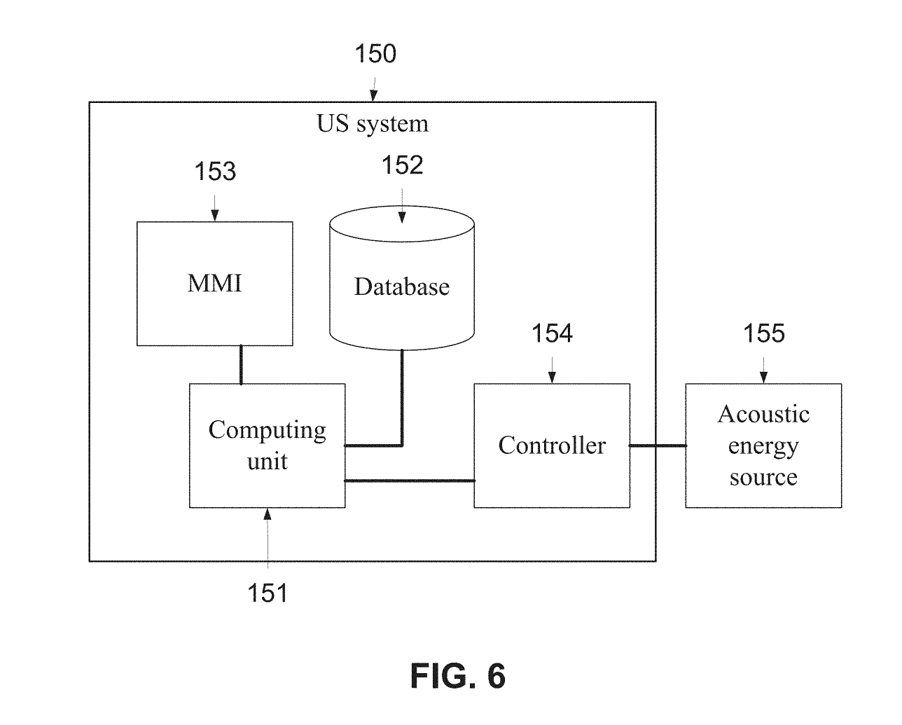

As shown at 102, one or more characteristics of the target tissue, membranous delivery vessel and/or surrounding biological tissues are provided. For brevity, reference to the of target tissue may be a reference to the characteristics of one or more membranous delivery vessels and the characteristics of surrounding biological tissues may be the characteristics of surrounding biological tissues at the target bodily region and/or organ. Optionally, these characteristics may be manually provided by a system operator via a man machine interface, such as a keyboard. Optionally, the MMI is part of a system that applies acoustic energy for changing the volume of intra bilayer membrane space of cells of a target tissue, for example as depicted in FIG. 6 and described below. Optionally, the system presents a user interface (UI) that allows the user to input these characteristics.

The characteristics of the target tissue, for example characteristics of the bilayer membrane of the target tissue, may include:
1. Trans-membrane proteins—an estimate of the presence or absence of trans-membrane proteins in the bilayer membrane 200, for example as shown at 206 of FIG. 2, the prevalence thereof and their gripping effect.
2. An areal stiffness of the bilayer membrane and of one leaflet (10.
3. A bending stiffness of the bilayer membrane and of one leaflet.
4. Aqueous solution characteristics—characteristics of the surrounding aqueous solution which surrounds at least some of the target tissue.
5. Mechanical properties of the surrounding tissue—properties of one or more surrounding tissues, for example elasticity and/or loss modulus, thickness and the like. For instance, thickness of the tissue layer above the intramembrane space is taken into account. The location of the target tissue in relation to surrounding tissues—one or more surrounding tissues may apply resistance force against the expansion of the intra-bilayer membrane space. For example, if the US frequency is 1 MHz, the shear modulus of the surrounding tissue is predicted to increase above 1 MPa. A layer of a surrounding tissue having thickness of 0.6 μm reduces the volume increment of the intramembrane space by 9-fold. This may explain why cells at a free surface such as the endothelial cells in blood vessels, are more susceptible to US than cells deep below the free surface. A much stiffer “tissue” may suppress the volume increment of intra-bilayer membrane space of adjacent cells in the same manner. The less is the applied resistance, the more susceptible to acoustic energy is the bilayer membrane 201.
6. The temperature of the target tissue and/or surrounding tissues.
7. The attraction/repulsion forces between the leaflets of the bilayer membrane.
8. Characteristics of a nearby single microbubble and/or microbubbles cloud, including their size distribution and density.

Now, as shown at 103, an acoustic energy transmission pattern is selected and/or calculated according to the one or more provided characteristics and/or one or more desired bioeffects. As used herein, an acoustic energy transmission pattern means a set of instructions for operating an acoustic energy source to generate one or more acoustic energy transmissions, optionally sequentially or simultaneously. The acoustic energy transmission pattern optionally defines the characteristics of each acoustic energy transmission, for example its amplitude, frequency and/or phase.

The acoustic energy transmission pattern optionally defines interludes between the transmissions. Optionally, the acoustic energy transmissions are emitted in a plurality of transmission cycles. The acoustic energy transmission pattern defines one or more transmission characteristics of acoustic energy for transmission. The transmission characteristics may be, for example, amplitude, a frequency, a transmission power, a transmission angle, the size of the focused beam, the spatial distribution of the acoustic field, a transmission interlude and/or any other characteristic which may change the effect of the acoustic energy on the volume of the intra-bilayer membrane hydrophobic space 201. An acoustic energy transmission pattern may be set to induce one or more bioeffects, for example creating ruptures in the cell's bilayer membrane for introducing exogenous material into the intra cellular space, stimulating and/or inhibiting cellular processes, and/or changing the mechanical characteristics of the cell.

Optionally, a database of acoustic energy transmission patterns is used. The database optionally includes a plurality of target tissue records. Each record defines an acoustic energy transmission pattern recommended to be applied to affect a bilayer membrane 201 of a biological tissue having one or more characteristics. Optionally, different patterns may be defined for different bioeffects on the target tissue, for example creating ruptures, changing mechanical characteristics, and stimulating and/or depressing cellular processes. Each record is associated with a different set of cellular characteristics, allows matching a suitable pattern to a biological tissue having cells with these cellular characteristics. Each acoustic energy acoustic energy transmission pattern has certain transmission characteristics, for example the amplitude(s), the frequency(ies), the power, the transmission angle, the transmission interlude(s) and/or any other transmission characteristic which may change the effect of acoustic energy on the volume of the intra-bilayer membrane hydrophobic space 201.

Each acoustic energy acoustic energy transmission pattern may be defined as a function of time where one or more transmission characteristics of the acoustic energy, for example the amplitude and/or the frequency, change over time. Each acoustic energy acoustic energy transmission pattern may define a plurality of acoustic energy transmission cycles.

The acoustic energy applies acoustic pressure at least on the bilayer membrane 200. Optionally, the acoustic pressure, which may referred to herein as a separating pressure and/or pressure, is applied so as to take apart two phospholipids leaflets of the bilayer membrane 200 and increases the volume therebetween. The separating pressure may be calculated as described by Jacob N. Israelachvili, Intermolecular and Surface Forces, Second Edition: With Applications to Colloidal and Biological Systems (Colloid Science), www(dot)amazon(dot)com/Intermolecular-Surface-Forces-Second Applications/dp/0123751810—#The calculation approximates the different forces expected to appear between two phospholipid bilayers, for example the attraction van der Waals (VDW) force between the leaflets 202, 203, repulsive forces, such as undulation and peristaltic forces which are associated with instability of thermal surface waves in the bilayer membranes, and protrusion forces. For example, when the distance between the leaflets 202, 203 is 1 nm to 2 nm and the leaflets are of a phospholipid bilayer membrane at 25° C., the calculation predicts pressures of attraction and repulsion and pressures of protrusion of less than about 0.1 MPa (10⁵ Pa).

Attraction and repulsion pressures between the leaflets 202, 203 are expected to be about the same as in between two bilayers, for example as described in Jacob N. Israelachvili, Intermolecular and Surface Forces, Second Edition: With Applications to Colloidal and Biological Systems (Colloid Science), which is incorporated herein by reference.

Optionally, the pattern selection is performed in accord with measurements on the force between two surfactant coated silica surfaces, for example see Sens, P. and S. A. Safran (1998). “Pore formation and area exchange in tense bilayer membranes.” Europhysics Letters 43(1): 95-100, which is incorporated herein by reference.

Optionally, the pattern selection is performed according to a desired increment to the volume of the intra-bilayer membrane space. The intra-bilayer membrane space 201 may be measured by a model having a maximum area strain ε_{A,max} where ε_{A}=(S−S₀)/S₀, and where S denotes a surface area of a deformed leaflet, such as 302 in FIG. 3. The model predicts that roughly ε_{A,max}∝P_{A}^{0.8}/kₛ^{0.5} (not shown). The model may be rather simple and therefore portrays an intra-bilayer membrane space 201 on a free surface, where the aqueous solution above the leaflets 202, 203 is not bound, namely the effect of surrounding tissues on ε_{A,max} is neglected, and the aqueous solution inertia is the main external force resisting the intra-bilayer membrane space 201 expansion. The effect of surrounding tissue may be incorporated in the model as greater kₛ that increases by adding 2Gd where kₛ and 2Gd are defined as in Boal, D. (2002). Mechanics of the Cell. New York, Cambridge University Press, which is incorporated herein by reference, G denotes the dynamic shear modulus of a cell and G=\sqrt{G′²+G″²} where G′ and G″ denotes elastic and loss modulus, and d denotes tissue thickness. For f=1 MHz G is predicted to go above 1 MPa, for example see Fabry, B., G. N. Maksym, C. Franks, et al. (2001). “Scaling the microrheology of living cells.” Physical Review Letters 87(14)(1976). “Stimulation of Healing of Varicose Ulcers by Ultrasound.” Ultrasonics 14(5): 232-236 (hereinafter: “Fabry and Maksym, 2001”), which is incorporated herein by reference. For d=0.6 μm, the value of ε_{A,max} in the first case, is reduced about nine folds.

Optionally, the pattern selection includes determining the amplitude of the applied acoustic energy. For example, when the amplitude is of about 0.1 MPa, it is capable of separating the two leaflets 202, 203 having a maximal attraction pressure of e.g. 0.014 MPa.

Optionally, the pattern selection includes determining the frequency of the applied acoustic energy. The effect of the acoustic energy on leaflet 202 is affected by the frequency of the acoustic energy. For example, different leaflets 202, 203 may vibrate in response to different frequencies.

Optionally, the pattern selection includes determining a number of frequencies for the acoustic energy. In use, the different frequencies may be transmitted simultaneously and or sequentially, for example using a multi transducer US probe and/or an ultrasonic phased array, an array of single ultrasound transducers each of which may be activated in a different fashion. For example, one of the frequencies is selected as a rectified diffusion transmission which is set to induce a leaflet motion is responsible for a gradual intra-bilayer membrane space growth and therefore to a gradual stretching of one or more of the leaflets 202, 203.

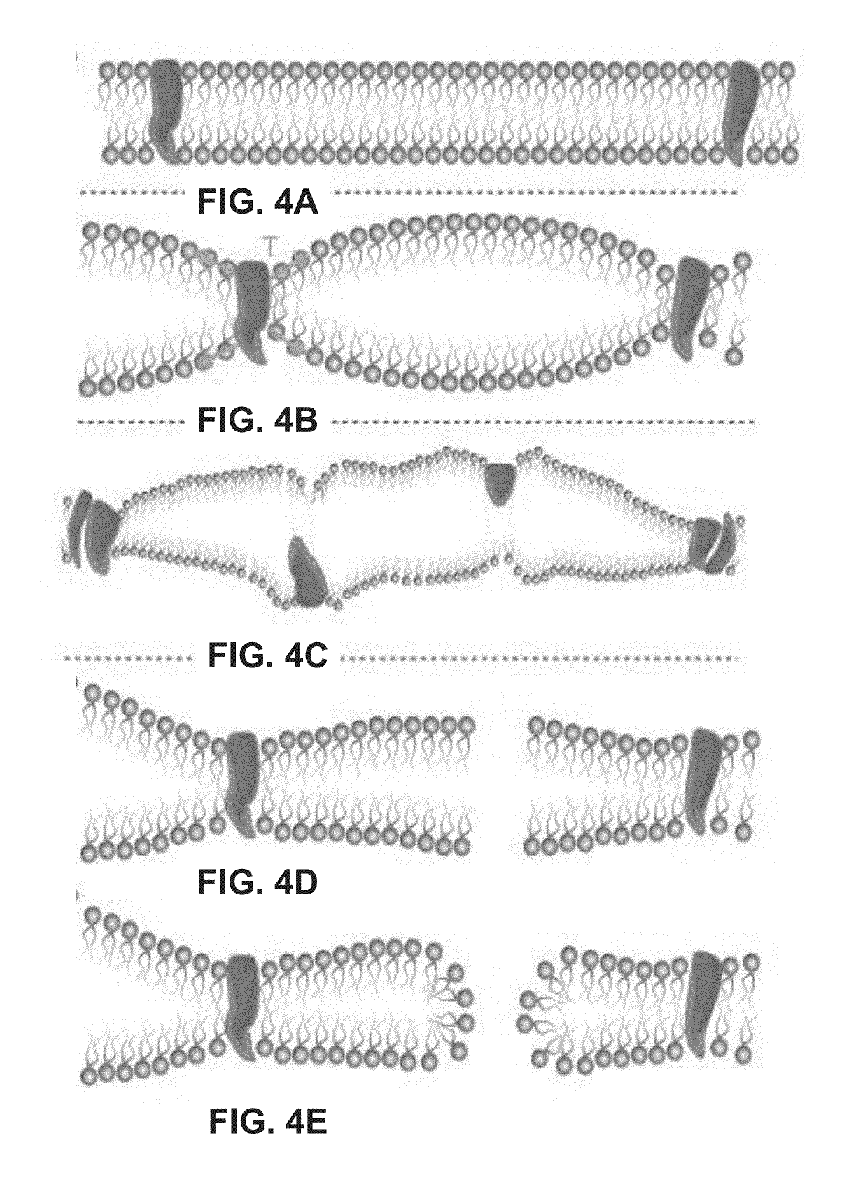

Optionally, the pattern selection includes calculating one of more growth interruption events and selecting a pattern which induces a desired growth interruption event. The growth interruption events may be reaching a maximal intra-bilayer membrane space volume where an increment in pressure does not induce an increment in volume, where one of the leaflets breaks open and the tension reaches a rupture threshold, and/or where the tension applied on the transbilayer membrane proteins is high enough to tear the leaflet away from the protein molecule, for example as shown at FIGS. 4D and 4E.

Optionally, the pattern selection includes takes into account cavitation safety limits. The volume is increased until the leaflets 202, 203 are stretched beyond some critical maximum ε_{A,max} which corresponds to a cavitation safety limit. At frequency above 20 kHz G˜G″∝f, as set in Fabry and Maksym, 2001, ε_{A,max}∝P_{A}^{0.8}/f^{0.5} is predicted whereas for US safety it is common to use a Mechanical Index (MI) which fulfills MI∝P_{A}/f_{0.5}, as defined in Barnett, S. B., G. R. Terhaar, et al. (1994). “Current Status of Research on Biophysical Effects of Ultrasound.” Ultrasound in Medicine and Biology 20(3): 205-218, which is incorporated herein by reference, a food and drug administration (FDA) cavitation threshold safety limit is used where MI=1.9. This limitation defines pressure, frequency, and proper coefficient thresholds for a human body, see Abbott, J. G. (1999). “Rationale and derivation of Mi and Ti—A review.” Ultrasound in Medicine and Biology 25(3): 431-441, which is incorporated herein by reference. Above this cavitation threshold, hemorrhage appears as a first sign of tissue damage, whereas it reflects rupture of endothelial cells.

Optionally, MI is kept below about 1.9 to prevent hemorrhage.

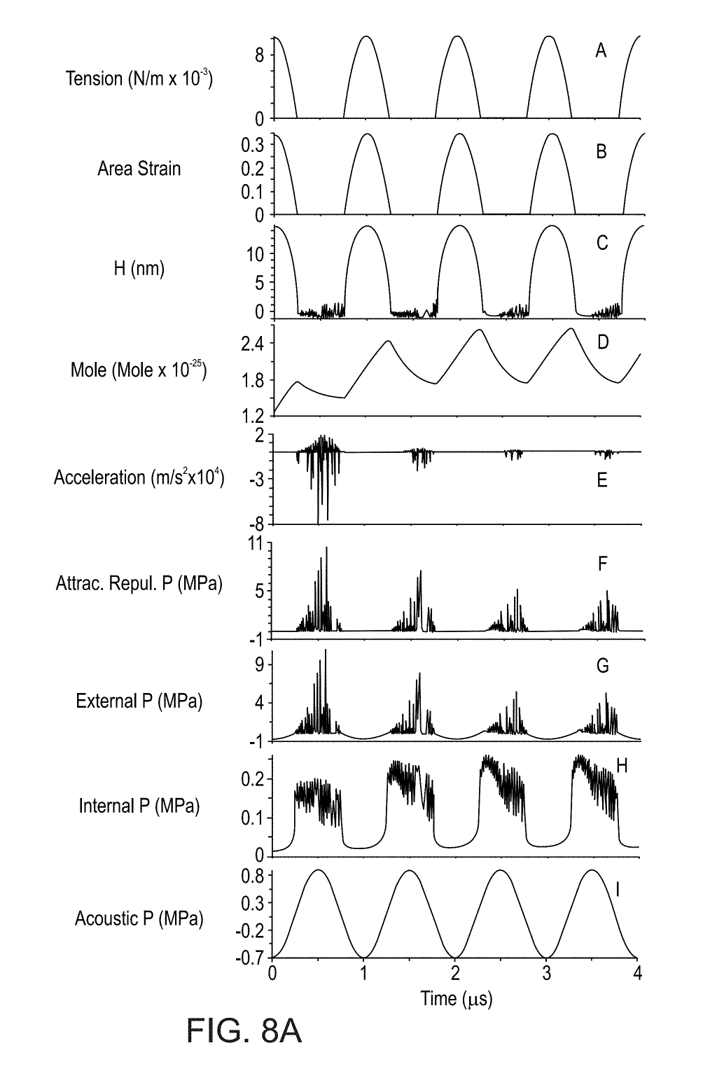

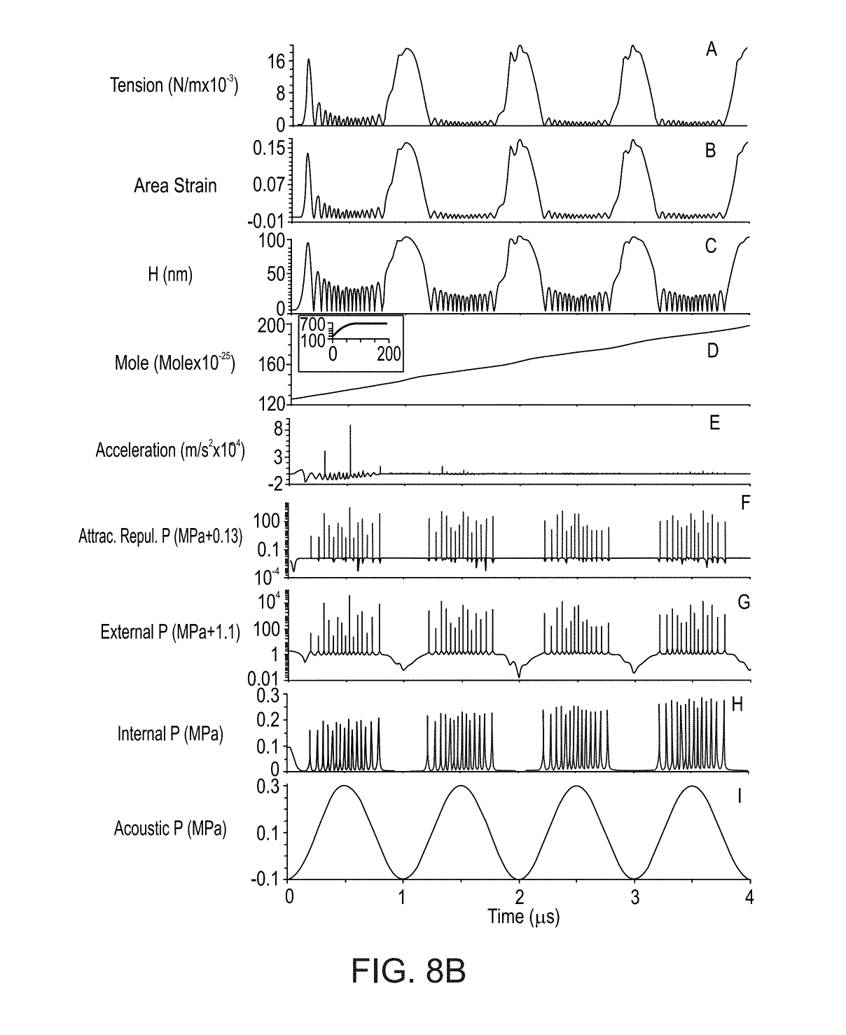

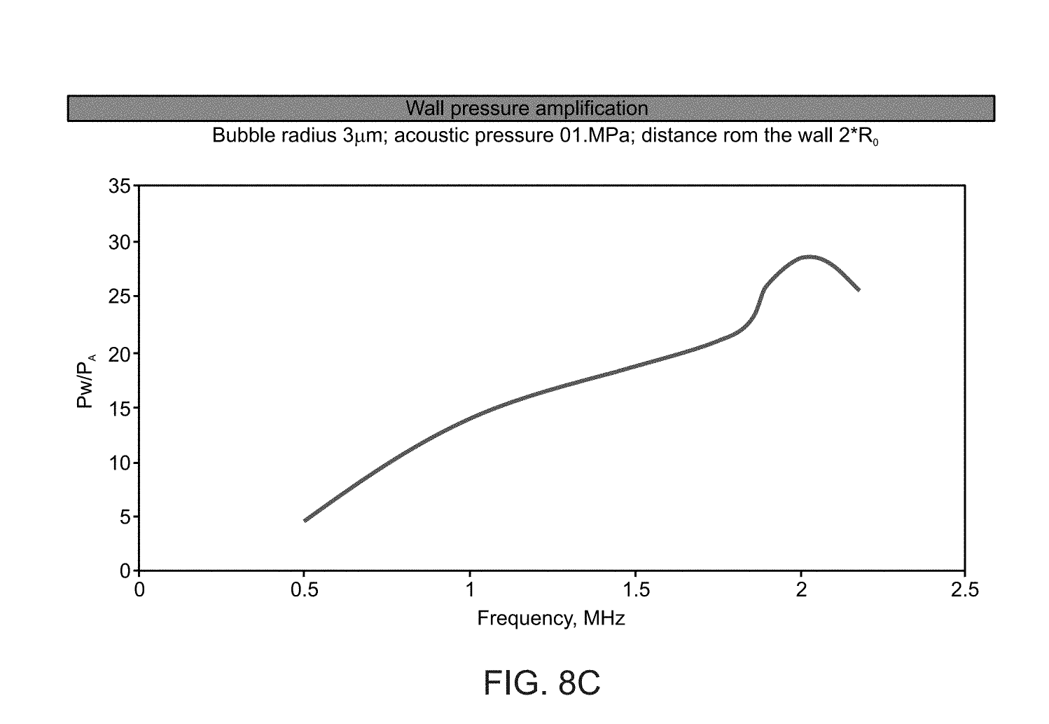

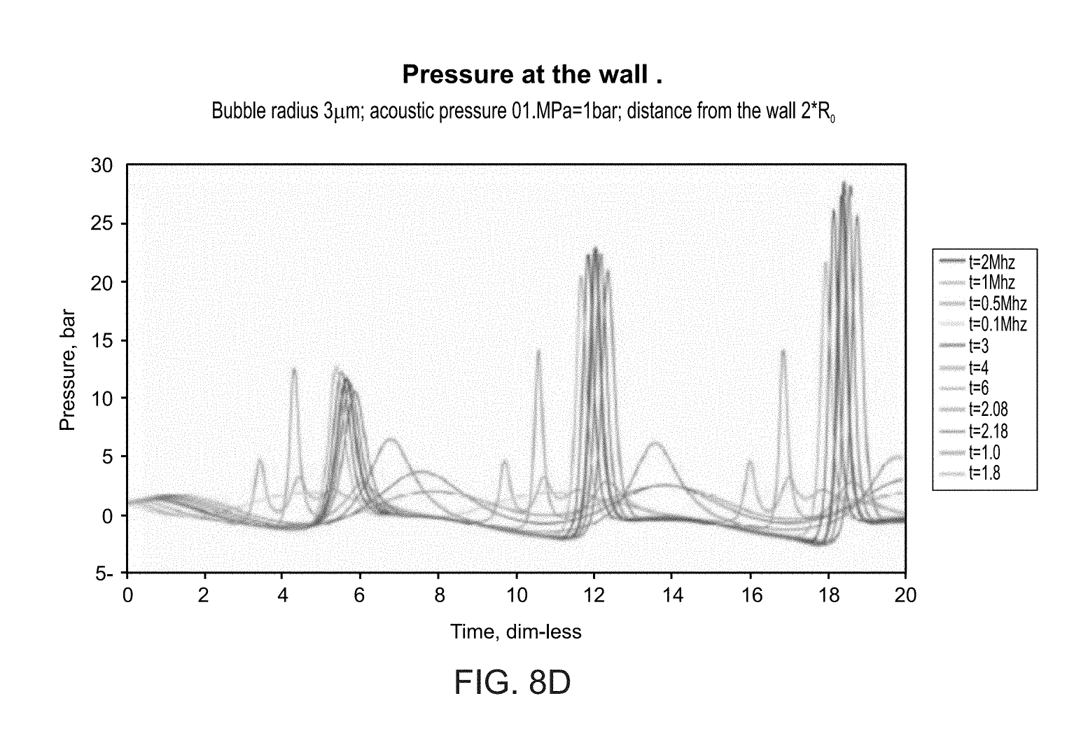

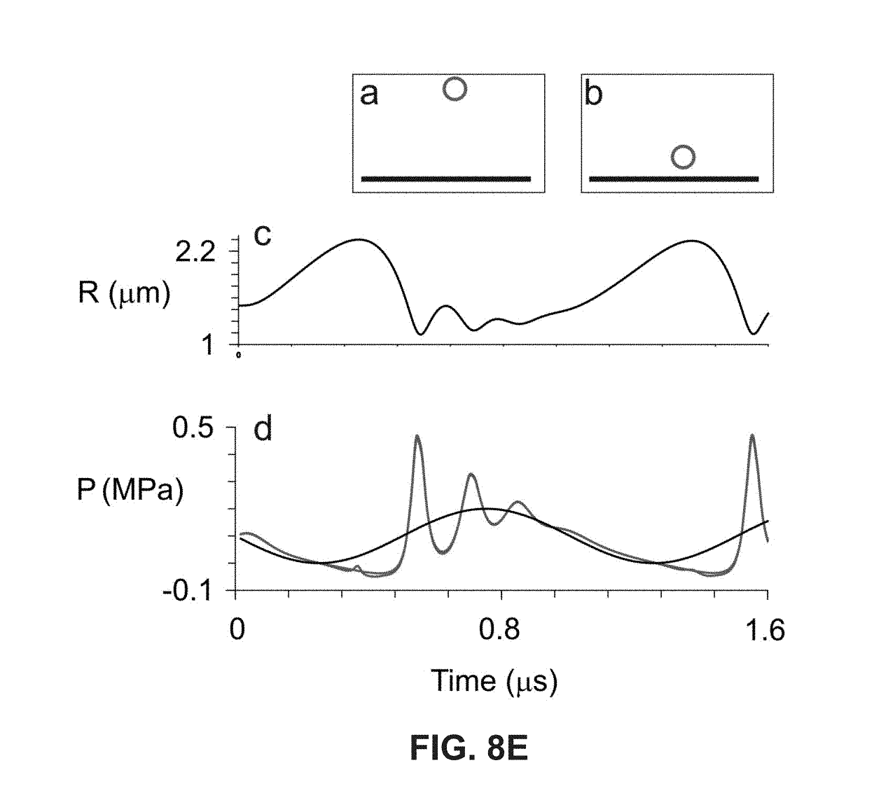

According to some embodiments of the present invention, an acoustic energy acoustic energy transmission pattern is calculated so as to increase the volume of a pulsating gas bubble in US field. Optionally, the calculation is based on a model of a bubble that steadily pulsates near a wall in ultrasonic field. For simplicity a spherical symmetry is assumed for the bubble. The bubble dynamics is optionally described by a Rayleigh-Plesset (RP) equation. A potential flow field is solved by Bernoulli energy conservation equation assuming the fluid around the bubble to be incompressible and non viscous. For example, a bubble having a diameter of 6 μm is placed 6 μm from the model wall, in a US field with pressure amplitude of 10⁵ Pa at infinity. On the model wall, just below the bubble, the pressure amplitude is estimated to increase up to about 30 times when the US frequency is about 2 MHz—the resonance frequency of the bubble, for example as shown at FIG. 8C.

According to some embodiments of the present invention, an acoustic energy acoustic energy transmission pattern is set to affect certain cells while avoiding applying any influence on neighboring cells. Some of the cells may be affected while several micrometers away a neighboring cell remains unaffected. This exemplifies the dominance of the intra-bilayer membrane over extracellular bubbles as the source of the observed bioeffects.

Now, as shown at 104, acoustic energy source is directed toward a target tissue. Optionally, the direction is set according to the selected pattern. Optionally, the direction is changed during the acoustic energy transmission process.

Optionally, the acoustic energy source is directed by one or more actuators, such as linear or rotary actuators, which are set to move the acoustic energy source 155 in relation to the target tissue according to the selected acoustic energy transmission pattern.

As shown at 105, one or more acoustic energy sources are instructed to apply acoustic energy on the target tissue according to the selected acoustic energy transmission pattern. By applying acoustic energy according to a pattern selected to match the characteristics of the biological tissue and/or the surrounding biological tissues, the volume of the intra bilayer membrane space is changed, optionally increased.

When the acoustic energy is applied, as described above, the atmospheric pressure may be zero and accordingly the acoustic pressure oscillates between positive values, when the pressure pushes water molecules closer to each other and negative values when the pressure pulls water molecules away from one another, against cohesion forces. At a negative pressure, the two leaflets 202, 203 are pulled away from one another, overcoming molecular attraction forces of about 10⁵ Pa or less, between them, inertia of water at close proximity to the bilayer membrane 201, and/or viscous forces. For brevity, it should be noted that bending resistance of the leaflet 202 is neglected for simplicity. The leaflets 202, 203 are clutched together trans-membrane proteins, for example as described below.

For example, FIG. 3 depicts an intra-bilayer membrane space 201 with an increased volume between the leaflets 202, 203. The increment in the volume of the intra-bilayer membrane space 201 detaches the leaflets from one another 202. It should be noted that the leaflet detachment may not be uniform. As shown at 204, trans-membrane proteins 204 clutch the leaflets 202, 203 to one another, changing the attraction force along leaflets of the bilayer membrane 201.

When one of the leaflets 302 is arched and another 301 is fixed, as shown at FIG. 3, the arched leaflet acquires a dome shape. For example, two exemplary cases are provided. In the first, the diameter of the bilayer membrane is 50 nm, the area compression modulus of a leaflet (kₛ) is about 0.03 N/m, and the acoustic energy applies an acoustic pressure of about 0.8 MPa. In the second, the bilayer membrane diameter of 500 nm, kₛ is about 0.12 N/m, and the applied acoustic pressure is about 0.2 MPa. Once the cells having these exemplary bilayer membranes are exposed to respective acoustic energy; the intra-bilayer membrane space 201 turns into a mechanical oscillator, and a source of cavitation activity. Similar to a gas bubble, the intra-bilayer membrane space 201 transforms the acoustic pressure into relatively large periodic displacements, magnifies the pulsating pressure in a liquid phase around it. Optionally, the acoustic energy is applied in a plurality of cycles. From the first cycle, the leaflets 202, 203 are detached and a dome shape intra-bilayer membrane space is generated, for example as shown in FIG. 3. In the first and second cases, the maximum deviation of the dome apex from the base of about 15 nm and 100 nm, denoted in FIG. 3 as H. The volume increment induces large areal strain in the pulsating leaflet 302 and the tension rises to substantial level order of about 0.01 N/m that is high enough to rupture the pulsating leaflet 302.

The response of the intra-bilayer membrane space 201 to the applied acoustic pressure is instantaneous and besides the dome apex deviation also tension in the leaflet 301 and areal strain oscillate at the acoustic pressure frequency; all reaching maximum amplitude from a first cycle after onset of US. The oscillations in internal gas pressure and the gas content reaches stable amplitude are a number of acoustic energy cycles. It should be noted that the intra-bilayer membrane space may reach a maximal size during any of the acoustic energy cycles, including the first. It should be noted that the apex deviation may be limited by opposing tension forces, for example surrounding cells pressure. High amplitude, high frequency pressure pulses are generated in the aqueous solution around the intra-bilayer membrane space 201 when the aqueous solution is brought to a sudden halt. At the same time, large acceleration pulses and repulsion strong forces, in peaks, are induced in the aqueous solution between the leaflets 202, 203. Natural frequencies about ten and even hundred times greater than the US frequency are developed in the first and second cases, achieving resonance conditions once the US frequency is properly chosen.

This process reverses at positive acoustic pressure and the motion of the leaflets 202, 203 may be determined by a dynamics force (pressure) balance equation that is based on Rayleigh-Plesset (RP) equation for spherical bubble dynamics, see, Leighton, T. G. (1997), the Acoustic Bubble, San Diego—London, Academic Press, which is incorporated herein by reference.

The applied pressure changes the rate of transport of dissolved gas from the surrounding aqueous solution to the intra-bilayer membrane space 201 and/or from the intra-bilayer membrane space 201 to surrounding aqueous solution as it causes the leaflet 302 to expand and/or contract periodically. This may be modeled by a diffusion equation.

Reference is now made to FIG. 6, which is a schematic illustration of a system that applies acoustic energy for changing the volume of intra bilayer membrane space of cells of a target tissue, according to some embodiments of the present invention. The system 150 may be used for implementing the method described in FIG. 1. The system 150 includes a computing unit 151, such as a personal computer, a laptop, a tablet and a client terminal. The computing unit 151 is set to calculate and/or select an acoustic energy acoustic energy transmission pattern according to the characteristics of a target tissue and/or surrounding biological tissues. Optionally, the computing unit 151 includes or connected to a database 152, such as the aforementioned atlas. In such an embodiment, the acoustic energy transmission pattern may be selected from the database 152 according to the characteristics of the target tissue and/or surrounding biological tissues. Optionally, the computing unit 151 is connected to a man machine interface (MMI) 153, such as a keyboard, a mouse, and/or a touch surface and to a display. The MMI 153 allows manually inputting the characteristics of the target tissue and/or adjusting the selected acoustic energy transmission pattern. The computing unit 151 is connected to an acoustic energy source 155. The acoustic energy source 155, may be an US source, such as one or more ultrasound transducers, for example piezoelectric crystal based ultrasound transducers and an ultrasonic phased array and/or an acoustic shock waves generator, for example an electrical spark discharge and/or an acoustic shock waves generator. Optionally, the computing unit 151 is connected to a controller 154 that operates the acoustic energy source 155 to emit acoustic energy according to the transmission pattern. The controller 154 receives instructions from the computing unit 151 and translates them to activate the acoustic energy source 155. Optionally, the controller is connected to one or more actuators, such as linear or rotary actuators, which are set to move the acoustic energy source 155 in relation to a target area in which the target tissue may be positioned. In used the controller 154 receives instructions from the computing unit 151 and translates the instructions to activate the actuators so as to direct the acoustic energy source 155 to emit acoustic energy according to the acoustic energy transmission pattern.

As described above, the selected transmission pattern which applied on the target tissue may be selected to achieve one or more bioeffects.

Reference is now made to FIG. 4A is an exemplary bilayer membrane 400 and FIGS. 4B-4E are schematic illustrations of different bioeffects to the leaflets 402 of the bilayer membrane 400, according to some embodiments of the present invention. As described above, acoustic energy may be applied according to acoustic energy transmission patterns which are selected to have a different acoustic bioeffect on the target tissue. FIGS. 4B-4E are exemplary acoustic bioeffects which may be caused by different acoustic energy transmission patterns. Each acoustic bioeffect may require an acoustic energy transmission pattern with different frequency, amplitude, number of cycles, and the like.

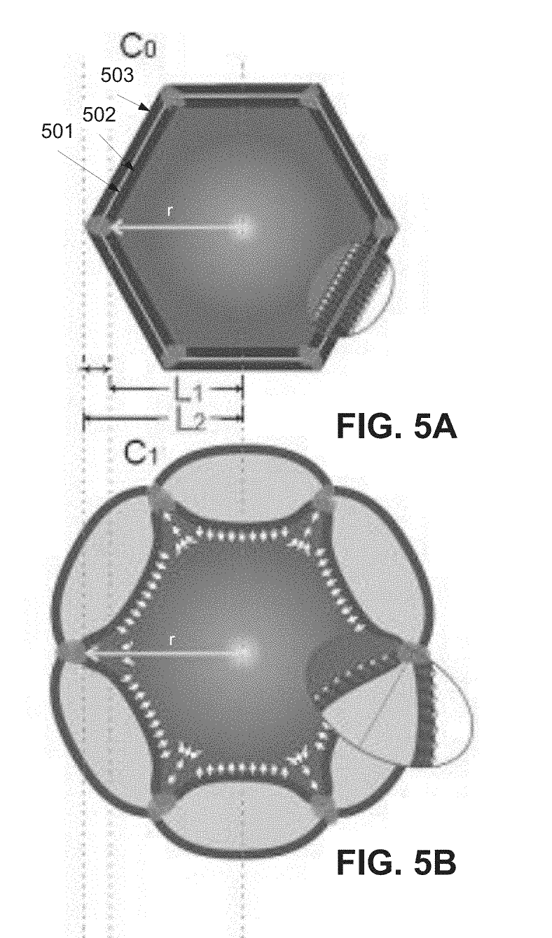

Optionally, the change in the volume of the intra bilayer membrane spaces 200 in the target tissue allows stimulating and/or unstimulating the target tissue. For example, when the desired acoustic bioeffect is a reversible and/or delicate bioeffect, for example as shown at FIG. 4B, an acoustic energy transmission pattern with a limited ε_{A,max} and/or low US intensity is applied. As shown at FIG. 4A the leaflets 402 are stretched and therefore may trigger the activation mechano-sensitive proteins in the bilayer membrane, which induce functioning change of cells sensitive to mechanical loading, such as endothelial cells, osteoblasts, fibroblasts, chondrocytes, and excitable cells. Cytoskeleton fibers may be stretched as well as shown in FIG. 5B.

Another exemplary bioeffect is depicted in FIG. 4C, which depicts a bioeffect based on the separation between the leaflets 402 and some of the trans-membrane proteins. In order to achieve such a bioeffect, an acoustic energy with greater than ε_{A,max} is applied. When this bioeffect is found, ruptures occur as an outcome of expanding the intra-bilayer membranes. As shown at FIG. 4C, stretching tension in the leaflets 402 disconnects the trans-membrane proteins from one of the leaflets 402. By disconnecting, the trans-membrane proteins are pulled out of the aqueous environment in the cell, outside the cell, or between lipid molecules of the leaflets 202, 203 and introduced into a gas pocket in an inner part of the bilayer membrane 400.

In excitable cells such as nerve cells or heart muscle cells, forming curved leaflets which are charged might result by polarization of the bilayer membrane, namely alterations of the electric field, and by dipole forming, see Petrov, A. G. (2006). “Electricity and mechanics of biobilayer membrane systems: Flexoelectricity in living bilayer membranes.” Analytica Chimica Acta 568(1-2): 70-83, which is incorporated herein by reference.

This polarization might induce ion flux across the bilayer membrane, not where both leaflets are separated by a gas filled intra-bilayer membrane, but rather in zones where both leaflets are still in contact and ion channels are functioning, as shown at FIG. 4B. Moreover, there is a possibility for a combined effect of dipole formation plus opening of mechano sensitive ion channels, for example as described in Casado, M. and P. Ascher (1998). “Opposite modulation of NMDA receptors by lysophospholipids and arachidonic acid: common features with mechanosensitivity.” Journal of Physiology-London 513(2): 317-33, which is incorporated herein by reference.

Additionally or alternatively, the volume change may allow introducing exogenous material into intra cellular space of cells via one or more hydrophilic passages formed in the intra-bilayer membrane hydrophobic space between the layers of the multi layered epithelium by the applied acoustic energy. In such an embodiment, the expansion of the intra bilayer membrane space stretches the leaflets 202, 203, forming ruptures that change the penetrability of the bilayer membrane 200. For example, FIG. 4D depicts a bioeffect in which the bilayer membrane 400 is perforated. The perforation may be performed by a spontaneous pore formation process at mildly stretched bilayer membranes, see Sens, P. and S. A. Safran (1998). “Pore formation and area exchange in tense bilayer membranes.” Europhysics Letters 43(1): 95-100, which is incorporated herein by reference. The perforation may be performed by bilayer membrane rupture when torn. The tension applied on the leaflets 202, 203 exceeds the rupture, for example when the intra-bilayer membrane pocket bursts open in one or more locations. Torn leaflets might fold and build a hydrophilic passage where water and larger molecules can pass from one side of the bilayer membrane 400 to another, for example as shown at FIG. 4E. Such passages may increase gene transfection rate, for example see Taniyama, Y., K. Tachibana, et al. (2002). “Local delivery of plasmid DNA into rat carotid artery using ultrasound.” Circulation 105(10): 1233-1239; Brayman, A. A., M. L. Coppage, et al. (1999). “Transient poration and cell surface receptor removal from human lymphocytes in vitro by 1 MHZ ultrasound.” Ultrasound in Medicine and Biology 25(6): 999-100; and Duvshani-Eshet, M., D. Adam, et al. (2006). “The effects of albumin-coated microbubbles in DNA delivery mediated by therapeutic ultrasound.” Journal of Controlled Release 112(2): 156-166.

Optionally, the formed passages enhance penetration of drug from the blood microcirculation into tissue across the endothelium. For example, the biological tissue is the blood brain barrier (BBB) and the formed passages enhance penetration of drug through. Optionally, the formed passages facilitate drug release from liposomes' enclosing bilayer membrane. Optionally, the formed passages facilitate enhanced delivery through the stratum corneum (SC).

Additionally or alternatively, the volume change may cause a complete irreversible damage to the bilayer membrane 400 for example or to cell necrosis, for example when the acoustic energy has a high intensity. The bioeffect in this case may be capillaries' hemorrhage triggered by ruptures in the bilayer membrane 400. Optionally, the target tissue includes cancerous cells and/or cells of capillaries which feeds cancerous cells, for example a tumor.

Additionally or alternatively, the change in the volume of the intra bilayer membrane spaces 200 in the target tissue allows changing mechanical characteristics of the target tissue.

Reference is a set of equations that allows defining the dynamics of an intra bilayer membrane space surrounded by an aqueous solution when an acoustic energy is applied thereon. These equations allow estimating the bioeffects of applying acoustic energy. Additionally, these equations allow estimating which acoustic energy has to be applied to achieve a desired bioeffect according to the characteristics of the target tissue. In such a manner, an acoustic energy transmission pattern may be selected or calculated, optionally automatically, according to these equations, in light of the characteristics of the target tissue.

Reference is now made FIGS. 5A and 5B, which are schematic illustrations of a simplified model of a cell and a cell with expended intra-bilayer membrane space. In the simplified model, a circular piece of a bilayer membrane, axisymmetric, is made of two parallel monolayer leaflets with zero force between then. The module may be used to calculate the acoustic energy transmission pattern.

A thin gas layer 501 compartment lies in between the two leaflets 502, 503 and aqueous solution that contains some dissolved gas fills the space that surrounds the upper leaflet 503. The lower leaflet 502 is fixed and cannot move. The rims of the leaflets are connected at the radii by a circumferential support that prevents any in plane motion. Uniform acoustic pressure (P_{A}) is applied toward the surface of the upper leaflet while attraction/repulsion force per area (pressure) is applied between the two leaflets 502, 503 from below. These forces may be parallel but not uniform. It is obtained by integration over a distributed force that varies with a radial coordinate (r) and depends on the local distance between the two leaflets. In addition, the pressure in the gas compartment is applied from below the leaflet. Due to force imbalance on the upper leaflet, it deforms perpendicular to the plane and acquires a dome shape as shown in FIG. 3.

When the deviation of the dome center from the initial planar position is small, for example H<Hₘᵢₙ, the mechanical response, for example acceleration, of the upper leaflet 203 and the aqueous solution 205 thereabove is negligible and the equilibrium equation is as follows:

Pₐᵣ+Pᵢₙ−P₀+P_{A} sin ωt=0 Equation 1:

where P_{A} denotes acoustic pressure, co denotes angular frequency of acoustic energy which is externally applied on the bilayer membrane 200, and Pₐᵣ denotes an attraction/repulsion pressure which is internally applied on the bilayer membrane 200 and may be defined as follow:

$$\begin{matrix}{P}_{ar} = \frac{2}{( {a}^{2} + {H}^{2} )} {\int }_{0}^{a} f ( r ) rdr . & Equation 2 \\ where f ( r ) denotes \text{:} & \\ f ( r ) = {A}_{r} [ {( \frac{σ}{h ( r ) + Δ} )}^{m} - {( \frac{σ}{h ( r ) + Δ} )}^{n} ] & Equation 3\end{matrix}$$

where Δ denotes an initial gap between the upper and lower leaflets 202, 203 h(r) denotes a local deviation of the leaflet 203 from its initial position.

It should be noted that the acoustic pressure (p) required to inflate a bubble overcomes the inward, contracting surface forces p˜2σ/r where σ denotes the surface tension and r denotes the bubble radius. For example, when r=1 nm, the required pressure amplitude exceeds 1.4·10⁸ Pa.

The local deviation h(r) may be expressed as follows:

h=\sqrt{R²−r²}−R+H. Equation 4:

where R denotes an instantaneous radius of the curved bilayer membrane and represented as follows:

$$R = \frac{{a}^{2} + {H}^{2}}{2 H} \qquad Equation 5$$

Pressure of the gas between the bilayer membrane and a solid Pᵢₙ is affected by the shape of the bilayer membrane 200. Assumed that in initial time moment Pᵢₙ=P₀ and depending on value of H may be expressed as:

$${P}_{in} = {{P}_{0} [ 1 + \frac{H}{6 Δ} ( 3 + \frac{{H}^{2}}{{a}^{2}} ) ]}^{- κ} \qquad Equation 6$$

where κ denotes a polytropic constant, which depends on the value of the gas volume and falls in interval between 1 and ratio of the gas specific heats. Taking into account the volume of the gas in this case, which is assumed κ=1. It is also assumed that in the initial moment t=0, when H=0 and Δ=s, the bilayer membrane is in equilibrium, namely Pₐᵣ=0.

These equations allows calculating (Equation 2÷Equation 6) and are substituted them into Equation 1 to provide a transcendental, quasi steady equation that may be solved for H(t). When H increases, the mechanical response of the leaflet 203 and the aqueous solution 205 cannot be neglected taken into account by using the following equations:

$$\begin{matrix} For H > {H}_{min} \text{:} \\ \frac{{d}^{2} H}{{dt}^{2}} + \frac{3}{2 R} {( \frac{dH}{dt} )}^{2} = \frac{1}{{ρ}_{l} R} [ {P}_{in} + {P}_{ar} - {P}_{0} + {P}_{A} sin ω t - {P}_{st} ( R ) - {P}_{s} ( R ) - \frac{4}{R} \frac{dH}{dt} ( \frac{3 {δ}_{0} {μ}_{s}}{R} + {μ}_{l} ) ] . & Equation 7 \\ For H < - {H}_{min} \text{:} \\ \frac{{d}^{2} \vert H \vert}{{dt}^{2}} + \frac{3}{2 R} {( \frac{dH}{dt} )}^{2} = \frac{1}{{ρ}_{l} R} [ - {P}_{in} - {P}_{ar} + {P}_{0} - {P}_{A} sin ω t - {P}_{st} ( R ) - {P}_{s} ( R ) - \frac{4}{R} \frac{d \vert H \vert}{dt} ( \frac{3 {δ}_{0} {μ}_{s}}{R} + {μ}_{l} ) ] . & Equation 8\end{matrix}$$

where ρₗ denotes the density of aqueous solution 205, μₗ denotes the dynamic viscosity of the aqueous solution 205, μₛ denotes dynamic viscosity of the bilayer membrane and δ₀ denotes initial thickness of the bilayer membrane 200.

The pressure Pₛ attributed to the circumferential tension per unit length (T) in the bilayer membrane may be found from the force balance:

$$\begin{matrix}T = {P}_{s} \frac{{a}^{2} + {H}^{2}}{4 H} . \\ where & Equation 8 \\ {P}_{s} = \frac{2 {k}_{s} \vert H {\vert}^{3}}{{a}^{2} ( {a}^{2} + {H}^{2} )} . & Equation 9\end{matrix}$$

where the area compression modulus of a leaflet

$${k}_{s} = \frac{E {δ}_{0}}{2 ( 1 - μ )} , \qquad Equation 10$$

is connected with the elasticity modulus E and the Poisson's ratio μ.

The area compression modulus (area stiffness) varies over a wide range between values lower than kₛ=0.06 N/m. An overestimated average value for a highly nonlinear curve of τ-S typical of undulated bilayer membrane at low tension, see Evans, E. and W. Rawicz (1990). “Entropy-Driven Tension and Bending Elasticity in Condensed-Fluid Bilayer membranes.” Physical Review Letters 64(17): 2094-2097 and Boal, D. (2002). Mechanics of the Cell. New York, Cambridge University Press, which are incorporated herein by reference, and kₛ=0.24 N/m for a stretched bilayer membrane, already flattened, see Phillips, R., T. Ursell, et al. (2009). “Emerging roles for lipids in shaping bilayer membrane-protein function.” Nature 459(7245): 379-385, which is incorporated herein by reference.

At low projected areal strain below some 10%, the leaflet is wavy and undulated, see Sens, P. and S. A. Safran (1998). “Pore formation and area exchange in tense bilayer membranes.” Europhysics Letters 43(1): 95-100, which is incorporated herein by reference. Stretching the leaflet in this case is primarily flattening it overcoming bending resistance; where the bending stiffness of a bilayer membrane is about 0.08 N/m (20 kBT, kB is the Boltzmann constant), and is 0.01 N/m for a half thickness leaflet, because bending stiffness ˜δ₀³. An upper limit for leaflet stretching stiffness that accounts both for stretching and bending is optionally set to the stretching stiffness of a bilayer membrane, for example 0.24 N/m or 60 kBT, see Phillips, R., T. Ursell, et al. (2009). “Emerging roles for lipids in shaping bilayer membrane-protein function.” Nature 459(7245): 379-385, which is incorporated herein by reference.

The diffusion of dissolved gas in the water is controlled by

$$\frac{\partial {C}_{a}}{\partial t} = {D}_{a} {∇}^{2} {C}_{a} \qquad Equation 12$$

where Cₐ denotes a mole concentration of air in the surrounding aqueous solution 205 and Dₐ denotes diffusion constant. The bilayer membrane 200 is a very small disc on a plane that bounds the space filled with water. No air diffuses through the plane and spherical symmetry is assumed. The initial and boundary conditions are:

Cₐ(ξ,0)=Cᵢₐ Equation 13:

Cₐ(a,τ)=Cₛ;τ>0 Equation 14:

According to Henry's law:

$${C}_{s} = \frac{{P}_{in}}{{k}_{a}} \qquad Equation 15$$

where kₐ denotes Henry's constant and the internal pressure, Pᵢₙ may be defined as follows:

$${P}_{in} = \frac{{n}_{a} {R}_{g} Ta}{{V}_{a}} . \qquad Equation 16$$

where R_{g} denotes a universal gas constant, Ta denotes an absolute temperature, and Vₐ denotes the air volume under the leaflet 203:

$${V}_{a} = π {a}^{2} Δ [ 1 + \frac{H}{6 Δ} ( 3 + \frac{{H}^{2}}{{a}^{2}} ) ] \qquad Equation 17$$

and the change of the air mole content under the membrane:

$$\frac{{dn}_{a}}{dt} = {{SD}_{a} ( \frac{\partial C}{\partial r} )}_{r = a} \qquad Equation 18$$

where S denotes a membrane surface and the initial condition of the equation is

$${{n}_{a} \vert}_{t = 0} = \frac{{P}_{0} {V}_{a}}{{R}_{g} T} . \qquad Equation 19$$

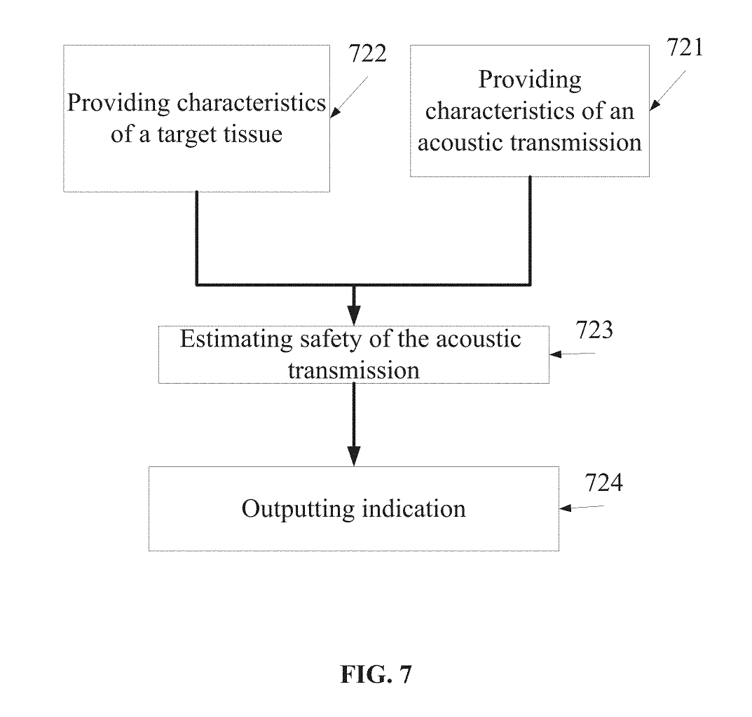

Reference is now made to FIG. 7, which is a method of estimating the safety of an acoustic energy transmission, according to some embodiments of the present invention. The set of equations 1-19 may be used for estimating the safety of an acoustic energy transmission having transmissions characteristics when applied on a target tissue having cells with certain characteristics, for example as defined above.

As shown at 721, one or more characteristics of cells of a certain target tissue are provided, for example as described in relation to numeral 102 of FIG. 1. As shown at 722, one or more characteristics of an acoustic energy transmission which is set to radiate the target tissue. The characteristics may include, for example, amplitude, a frequency, a transmission power, a transmission angle, the size of the focused beam, the spatial distribution of the acoustic field, a transmission interlude and/or any other characteristic which may change the effect of the acoustic energy on the volume of the intra-bilayer membrane hydrophobic space 201 of the acoustic energy transmission which is generated and transmitted on the cells of the target tissue. Optionally, the acoustic energy transmission is a transmission of an ultrasonic probe during an ultrasonic diagnosis, an ultrasonic treatment, and/or ultrasound-guided procedure.

Now, as shown at 723, the level of safety of the acoustic energy transmission is estimated. When acoustic energy is applied, safety is achieved by avoiding undesired bioeffect to the membrane of the cells such as cavitation, ruptures, pores, and/or any irreversible bioeffect, see Common US bioeffects in high US intensity include for instance lysis of red blood cells (RBC) in vitro, see Carstensen, E. L., P. Kelly, et al. (1993). “Lysis of Erythrocytes by Exposure to CW Ultrasound.” Ultrasound in Medicine and Biology 19(2): 147-165, which is incorporated herein by reference, damage to blood vessels and hemorrhage, see Child, S. Z., C. L. Hartman, et al. (1990). “Lung Damage from Exposure to Pulsed Ultrasound.”, which are incorporated herein by reference.

Optionally, the estimation is made based on an estimation of an increment in the volume of an intra-bilayer membrane space of the cells in response to the acoustic energy transmission. Such estimation may be based on the outcome of equations 1-19. Optionally, the estimation is performed according to cavitation safety limits. If the estimation is that the intra membrane volume is increased so that the leaflets 202, 203 are stretched beyond a threshold ε_{A,max} which corresponds to a cavitation safety limit, the estimation is that the acoustic energy transmission is not safe. For example the threshold may be defined at frequency above 20 kHz G˜G″∝f, as set in Fabry and Maksym, 2001, ε_{A,max}∝P_{A}^{0.8}/f^{0.5} is predicted. Optionally, the threshold is set for US safety and fulfills MI∝P_{A}/f^{0.5}, as defined in Barnett, S. B., G. R. Terhaar, et al. (1994). “Current Status of Research on Biophysical Effects of Ultrasound.” Ultrasound in Medicine and Biology 20(3): 205-218, which is incorporated herein by reference.

Optionally, the estimation is based the bioeffects induced by the acoustic energy transmission, for example the ruptures it creates in the cell's bilayer membrane, stimulating and/or inhibiting cellular processes, and/or changing the mechanical characteristics of the cell. The threshold for creating such bioeffect is described. Inter alia in relation to numeral 103 of FIG. 1 above.

Now, as shown at 724, an output indicative of the safety level is generated, an optionally presented to an operator. Such a method may be implemented by a system having an ultrasound probe for verifying its safety, a system for estimating safety of acoustic energy transmissions, and the like.

Reference is now made to another set of equations that defines the pressure amplification that is applied on the leaflets by a pulsating gas bubble. Similarly to the above set of equations, this set of equations allows estimating one or more bioeffects of a certain acoustic energy transmission pattern. In such a manner, an acoustic energy transmission pattern may be selected or calculated according to the characteristics of the target tissue, for example the characteristics of the bilayer membrane, and/or a desired effect, for example creating ruptures and/or pores in the layer membrane.

The following equations describe a bubble that pulsates steadily near a wall in ultrasonic field and acts as an amplifier of the acoustic pressure pulse. The bubble may amplify the pressure pulse even when not near a wall. The equations describe the dynamics of a bubble with a spherical symmetry, in spite of the presence of the wall near the bubble. Consider a spherical bubble in infinite space subjected to ultrasound field. The pulsations of the bubble are described by the following equation for bubble dynamics:

$$( 1 - \frac{\dot{R}}{{C}_{l}} ) R \ddot{R} + \frac{3 {\dot{R}}^{2}}{2} ( 1 - \frac{\dot{R}}{3 {C}_{l}} ) = ( 1 + \frac{\dot{R}}{{C}_{l}} ) \frac{P}{{ρ}_{L}} + \frac{R}{{C}_{l}} \frac{1}{{ρ}_{L}} \frac{dP}{d τ} \qquad Equation 20$$

where the initial condition is defined as follows:

$$\begin{matrix}{R \vert}_{τ = 0} = {R}_{0} & Equation 21 \\ and & \\ P = {P}_{L} - {P}_{∞} - \frac{2 σ}{R} - \frac{4 μ \dot{R}}{R} ; & Equation 22\end{matrix}$$

where P_{∞} denotes the pressure at infinity, oscillating with time:

P_{∞}=P₀[1+A Sin(ωt+β₀)];

ω=2πf; Equation 23:

In the adiabatic case, pressure inside the bubble P_{L} is represented in the following form:

$${P}_{L} = ( {P}_{0} + \frac{2 σ}{{R}_{0}} ) {( \frac{{R}_{0}}{R} )}^{3 κ} \qquad Equation 24$$

where τ denotes time, R denotes a bubble radius, and R₀ denotes the radius initial value;

$$\dot{R} ≡ \frac{dR}{d τ} ; \ddot{R} ≡ \frac{{d}^{2} R}{d {τ}^{2}} ; \qquad Equation 25$$

where P₀ denotes the initial pressure of the gas inside the bubble, P_{L} is the pressure inside the bubble, σ denotes surface tension, κ denotes the gas ratio of specific heats, μ denotes the dynamic viscosity of the liquid; ρL the liquid density, Cₗ the velocity of sound in the liquid, and f denotes the frequency of the acoustic energy.

The pressure distribution along the z-axis is derived from the energy conservation (Bernoulli) equation along a streamline of a non-compressible non-viscous liquid:

$$\frac{p}{{ρ}_{L}} + \frac{\partial θ}{\partial τ} + \frac{{v}^{2}}{2} = const . \qquad Equation 26$$

where θ denotes the velocity potential. Assuming, that Pₛ, the pressure at the bubble external surface, one gets an expression for the pressure at the wall:

$${p}_{w} = {P}_{s} - {ρ}_{L} {\int }_{H - R}^{0} [ \frac{\partial θ}{\partial τ} + \frac{1}{2} {( \frac{\partial θ}{\partial z} )}^{2} ] dz . \qquad Equation 27$$

The pressure at the bubble surface may be expressed as:

$${P}_{s} = {P}_{L} - \frac{2 σ}{R} . \qquad Equation 28$$

Potential flow solution may be obtained around a gas bubble which pulsates near a rigid wall in a non-viscous liquid. The equation for the velocity potential θ at time t may be written in the following form:

$${∇}^{2} θ ≡ \frac{1}{x} \frac{\partial }{\partial x} ( x \frac{\partial θ}{\partial x} ) + \frac{{\partial }^{2} θ}{\partial {z}^{2}} = 0 ( 2.9 ) \\ z \geq 0 ; - ∞ < x < ∞ ; {( z - H )}^{2} + {x}^{2} > {R}^{2} \qquad Equation 29$$

and the boundary conditions are defined as follow:

$$\begin{matrix}\frac{\partial θ}{\partial z} = 0 at z = 0 ; and & Equation 30 \\ \frac{\partial θ}{\partial n} = \dot{R} ( t ) at the bubble surface & Equation 31\end{matrix}$$

where n denotes an external normal to the bubble surface and R(t) denotes a solution of the bubble dynamic equation.

θ→0 at x→±∞ and/or z→∞; Equation 32:

It is expected that during the life of a patent maturing from this application many relevant methods and systems will be developed and the scope of the term US transducer, a computing unit, and a controller is intended to include all such new technologies a priori.

As used herein the term “about” refers to ±10%.

The terms “comprises”, “comprising”, “includes”, “including”, “having” and their conjugates mean “including but not limited to”. This term encompasses the terms “consisting of” and “consisting essentially of.

The phrase “consisting essentially of” means that the composition or method may include additional ingredients and/or steps, but only if the additional ingredients and/or steps do not materially alter the basic and novel characteristics of the claimed composition or method.

As used herein, the singular form “a”, “an” and “the” include plural references unless the context clearly dictates otherwise. For example, the term “a compound” or “at least one compound” may include a plurality of compounds, including mixtures thereof.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental support in the following examples.

### Examples

Reference is now made to the following examples, which together with the above descriptions; illustrate some embodiments of the invention in a non limiting fashion.

The following in vivo examples were carried out using a multi-layered epithelium model that have been previously evaluated for describing ultrasound induced bio-effects in Frenkel, V., E. Kimmel, et al. (1999). “Ultrasound-induced cavitation damage to external epithelia of fish skin.” Ultrasound in Medicine and Biology 25(8): 1295-1303; Frenkel, V., E. Kimmel, et al. (2000). “Ultrasound-facilitated transport of silver chloride (AgCl) particles in fish skin.” Journal of Controlled Release 68(2): 251-261; and Frenkel, V., E. Kimmel, et al. (2000). “Ultrasound-induced intercellular space widening in fish epidermis.” Ultrasound in Medicine and Biology 26(3): 473-480, which are incorporated herein by reference.

An epidermis of a fish, which lacks the SC of terrestrial vertebrates and resembles to a mucous bilayer membrane is used. This epidermis is located exteriorly to their scales and contains mucous secreting cells, which are analogous to goblet cells that migrate to the epidermal surface where they release their contents.

Common gold fish, 4-5 cm in length, were obtained from a nearby commercial fish farm, maintained in filtered fresh water at room temperature (20° C.), and fed ad libidum. Following an acclimation period of at least one week, treatments were carried out individually using the following procedure. Fish were placed in a 1 liter (L) holding tank containing the anesthetic benzocaine at a concentration of 0.25 gL⁻¹. Once they stopped swimming, they were removed from the tank and a 1.27 centimeter wide strip of foam rubber was secured around their mid section. This was then used fasten the fish to the bottom of a larger (12 L) tank filled with fresh tap water, also at room temperature. Ultrasound exposures were carried out using a standard physical therapy device branded Sonicator 720 of Mettler Electronics™ from California USA. The transducer of the device was inserted into the tank, just below the water line, where an active region of 10 cm² was positioned directly over the head of the fish and parallel to the space between the fish's eyes, at a distance of approximately 15 cm. Exposures were carried out in continuous mode at 1 and 3 MHz, and at a range of intensities (0.5-2.0 W cm⁻²) and durations (30-120 s). Exposures at 1 MHz, at all the intensities, generated acoustic cavitation in the fluid medium between the transducer and the treated surface see Frenkel, V., E. Kimmel, et al. (1999). “Ultrasound-induced cavitation damage to external epithelia of fish skin.” Ultrasound in Medicine and Biology 25(8): 1295-1303.

On the other hand, exposures at 3 MHz did not generate cavitation, even at the highest intensity used, which was still below the cavitation threshold, see Frenkel, V, E. Kimmel, et al. (2000). “Ultrasound-induced intercellular space widening in fish epidermis.” Ultrasound in Medicine and Biology 26(3). The presence or lack thereof of acoustic cavitation during the exposures was validated using both standard instrumentation (diagnostic ultrasound) and through ultra-structural alterations observed in processed samples (see below), appearing generally in the outer membranes of the surface cells.

Immediately after the exposures, the fish were taken out of the tank and a scalpel was used to remove a 3×3 mm section (0.5 mm thick) of the epidermis from the inter-eye region. Samples were fixed in glutaric dialdehyde (3% v/v), post-fixed in osmium tetroxide (1% v/v), both in sodium cacodylate buffer (0.1 M, pH=7.3), dehydrated in increasing concentrations of ethanol (50-100%), cleared with propylene oxide, and embedded in Epon (45% Agar 100 resin; 26.7% Methyl Nadia Anhydride; 26.7% Dodecenyl Succinic Anhydride; 1.6% Benzyldimethylamine v/v). Sections from the hardened blocks were cut perpendicular to the skin surface, mounted on copper grids, and then stained with both uranyl acetate and lead citrate. Representative micrographs of control and treated tissues were taken in black and white at magnifications ranging from 2,000 to 50,000 using a transmission electron microscope (JEM-100S, JOEL, Japan). These were subsequently scanned and saved digitally in JPEG format.

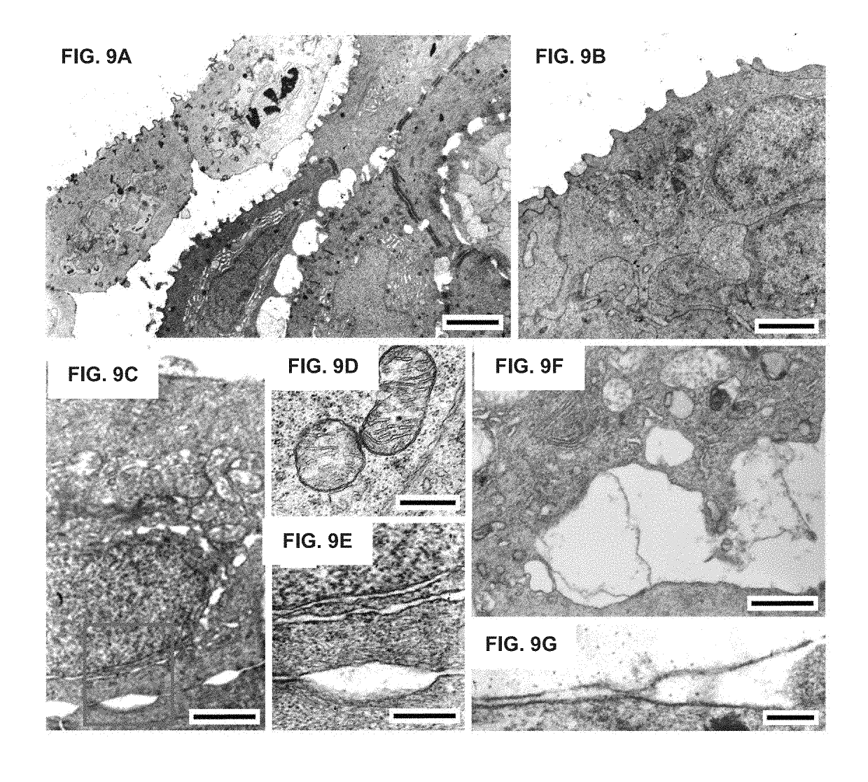

Reference is now made to FIGS. 8A and 8B which are graphs of transmission and biological tissue characteristics measured during four cycles of exposure to continuous wave (CW) acoustic energy. In FIG. 8A, the CW acoustic energy has a frequency 1 MHz and the biological tissue has cells with round membrane with a diameter 50 nm, as shown in FIG. 9A. The applied pressure has amplitude of 0.8 MPa. In this example the external leaflet is not stretched and kₛ=0.03 N/m. In FIG. 8B, the CW acoustic energy is applied on cells with a diameter of 500 nm and applied pressure has amplitude of 0.2 MPa. In this example the external leaflet is fully stretched and kₛ=0.12 N/m (˜30 k_{B}T Jnm⁻²). Plot A in FIGS. 8A and 8B depicts the tension force (T, N/m) in the moving leaflet. Plot B depicts the tension in the moving leaflet area strain. Plot C depicts the deviation (H, nm) of the dome apex. Plot D depicts Mole content (Moles·10⁻²⁵) in the intra membrane space between the leaflets. Plot E depicts acceleration (m/s²) of the aqueous solution above the moving leaflet. Plot F depicts an average attraction/repulsion force per area (Par, MPa) between the two leaflets. Plot G depicts external pressure (MPa) in the aqueous solution just above the moving leaflet. Plot H depicts internal gas pressure (Pi, MPa) in the intra space membrane between the leaflets. Plot I depicts an acoustic pressure (PA, MPa) far away from the leaflets.

Reference is now made to FIGS. 8C-8E which depicts an actual pressure pulse and amplification applied on a wall membrane by an exemplary bubble and the effect of the distance between the center of the bubble and the membrane wall, according to some embodiments of the present invention. When a bubble is formed at the bilayer membrane space, a pressure amplitude is estimated to increase up to about 30 times when the US frequency is about 2 MHz—the resonance frequency of the bubble, for example as shown at FIG. 8C. At the same time, the peak negative pressure decreases from zero Pascal to less than −0.1 MPa as shown FIG. 8D which depicts the pressure at the membrane wall during the first 3 cycles for various ultrasound frequencies. As depicted in FIG. 8D, the maximum negative pressure in absolute value is obtained at 2 MHz.

Reference is now made to FIG. 8E, which depicts, in a number of graphs and illustrations, the effect of the distance of the bubble from the membrane wall for a free bubble with equilibrium diameter of 3 μm that pulsates in US field with f=0.5 MHz and PA=0.1 MPa. The left box, marked with the letter a, depicts a distance of 12 μm between the bubble center and the membrane wall. The right box, marked with the letter b, depicts a distance of 2.36 μm between the bubble center and the membrane wall. The graph marked by c depicts the bubble radius (R) variations during a period. The graph marked by d depicts the bubble radius (R) variations c, when the pressure pulse is set at infinity (in black line) and the membrane wall is just below the pulsating bubble. When the at a distance between the bubble center and the wall is of 2.36 μm (in red line), and when the distance between the bubble center and the wall is 12 μm (in blue line).

Reference is now made to FIGS. 9A-9G which are images the bioeffects of acoustic energy transmissions on a fish skin tissue. FIG. 9A depicts the three outer layers of a fish skin 2 hrs after it is exposed to a cycle of acoustic energy transmission having a frequency of 1 MHz and a sequential cycle of acoustic energy transmission having a frequency of 3 MHz. The outer layers are necrosed, evident by compromised apical membrane and reduced electron density. Cells are also detaching from the second layer whose cells undergo differentiation to become surface cells (note micro-ridges already formed on their apical surface). Pocket-shaped gaps are observed between the second and third layer cells, and to a lesser extent between the third and the fourth layers, all of which are still viable. Bar=2 μm. In the cell on the left in the 2nd layer, intracellular gaps are also observed in the endoplasmic reticulum. Larger gaps are also observed where desmosomes are absent. FIG. 9B depicts outer layers of a control skin. The outer cells possess micro-ridges on their apical surfaces. Bar=2 μm. FIG. 9C depicts an outer cell immediately after receiving a 3 MHz exposure. Gaps are observed within the intercellular space between the surface cell and the cell immediately beneath it. Gaps are also visible at the nuclear membrane, being larger closer to the apical (upper) side of the cell. Bar=1 μm. FIG. 9D depicts an enlargement of box marked in FIG. 9C. Widening of the two nuclear membranes is shown at the upper part above pocket like gap between cells. Bar=0.5 μm. FIG. 9E depicts mitochondria in the second layer cell immediately after receiving a 3 MHz exposure. Disruption of the outer membrane is observed in the mitochondrion on the right, as well as some disruption of the cristae. The cristae in the mitochondrion on the left appear to be completely disrupted. Bar=0.5 μm. FIG. 9F depicts a gap between the first and the second layer cells immediately after receiving a 3 MHz exposure, where membrane sheets, some intact some not, bridge between the two cells. Some mitochondria in the outer cell appear to be completely disrupted. Bar=1 μm. FIG. 9G depicts widening of the apical membrane, with some ruptures, of a 2nd layer cell immediately after receiving a 1 MHz exposure. The outer layer cell has already sloughed off during the exposure. Bar=0.2 μm.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of changing the volume of an intra-bilayer membrane space of at least one bilayer membranous structure, comprising:
providing at least one characteristic of said at least one bilayer membranous structure;
selecting an acoustic energy transmission pattern set to change a volume of an intra-bilayer membrane space of a bilayer membrane of said at least one bilayer membranous structure according to said at least one characteristic; and
applying acoustic energy on the target tissue according to said selected acoustic energy transmission pattern.

2. The method of claim 1, wherein said at least one bilayer membranous structure is at least one cell, said providing comprising providing at least one characteristic of a target tissue having said target at least one cell.

3. The method of claim 1, wherein said at least one bilayer membranous structure comprises at least one membranous delivery vessel, said providing comprising providing at least one characteristic of a target tissue having said target at least one bilayer membranous structure.

4. The method of claim 1, wherein said at least one bilayer membranous structure is a member of a group consisting of a cell, a cell organelles, a membranous delivery vessel, a liposome, and any microorganism encapsulated by a bilayer membrane.

5. The method of claim 2, wherein said selecting is performed according to at least one desired bioeffect on the target tissue.

6. The method of claim 2, further comprising directing at least one acoustic energy source in front of the target tissue according to said selected acoustic energy transmission pattern and using said at least one acoustic energy source for performing said applying.

7. The method of claim 1, wherein said acoustic energy transmission pattern defines a plurality of sequential acoustic energy transmission cycles.

8. The method of claim 7, wherein each said acoustic energy transmission cycle, apart from the first of said plurality of sequential acoustic energy transmission cycles have a higher frequency than another said acoustic energy transmission cycle.

9. The method of claim 1, wherein said selecting comprises selecting at least one member of a group consisting of: a frequency of an acoustic energy transmission, a transmission power of said acoustic energy transmission, a transmission angle of said acoustic energy transmission, and a transmission interlude according to said at least one characteristic.

10. The method of claim 1, wherein said selecting estimating at least one of attraction force and repulsion force between leaflets of said intra-bilayer membrane.

11. The method of claim 1, wherein said selecting is performed according to a desired increment in the volume of the intra-bilayer membrane space.

12. The method of claim 1, wherein said selecting comprises estimating the volume of a pulsating gas bubble generated by acoustic energy transmission energy according to said at least one characteristic and selecting said acoustic energy transmission pattern according to said volume.

13. The method of claim 2, wherein said applying is performed to induce cell necrosis in said target tissue.

14. The method of claim 2, wherein said applying is performed to change a rate of introducing exogenous material into the intra cellular space of cells of said target tissue.

15. The method of claim 1, wherein said applying is performed to stimulate at least one cellular process in said target tissue.

16. The method of claim 1, wherein said applying is performed to slow down at least one cellular process in said target tissue.

17. The method of claim 2, wherein said applying is performed to change at least one mechanical characteristic of at least one bilayer membranous structure of said target tissue.

18. The method of claim 1, wherein a frequency of said acoustic energy is between 0.1 MHz and 30 MHz.

19. The method of claim 1, wherein an amplitude of a pressure applied by said acoustic energy on said bilayer membrane is about 0.1 megapascal (MPa).

20. The method of claim 1, wherein said volume is defined between transbilayer membrane proteins connecting leaflets of said bilayer membrane.
